(19) United States
(12) Patent Application Publication
(10) Pub. No.: US 2005/0261543 A1
(43) Pub. Date: Nov. 24, 2005

(54) Implantable artificial ventricular assist device

(76) Inventors: Yusuke Abe, Tokyo (JP); Takashi Isoyama, Tokyo (JP); Isao Nemoto, Chiba-Ken (JP)

Correspondence Address: PEACOCK MYERS, P.C., P O BOX 26927, ALBUQUERQUE, NM, 87125-6927, US

(21) Appl. No.: 11/133,063

(22) Filed: May 18, 2005

(30) Foreign Application Priority Data
May 18, 2004 (JP) 2004-176262

(51) Int. Cl. A61M 1/12

(52) U.S. Cl. 600/16

(57) ABSTRACT

The present invention provides an implantable, artificial ventricular assist device and system employing an undulation pump and methods for the use thereof. The undulation pump has a toroidal-shaped pump chamber with two angled side walls, an arc-shaped outer wall, an inlet port, an outlet port, and an inner circumferential opening and an undulation disk with a diameter that extends to about the arc-shaped outer wall of the pump chamber. The device includes a circumferential, flexible inner wall membrane covering the inner circumferential opening and least one surface of the undulation disk and forming liquid-tight seals to the pump chamber, or alternatively a precession assembly with inner bearings connected in series with an anti-rotation assembly, the anti-rotation assembly disposed within the pump and connected to the undulation disk, and preferably both, and a motor in connected communication with the precession assembly so that the disk undulates when motive force is applied.

### CROSS-REFERENCE TO RELATED APPLICATION

This application claims the benefit of the filing of Japanese Application No. 2004-176262 entitled “Implantable Artificial Heart”, filed May 18, 2004, and the specification and claims of that application are incorporated herein by reference.

### BACKGROUND OF THE INVENTION

1. Field of the Invention (Technical Field)

The present invention relates to an implantable artificial ventricular assist device to assist the heart function of a patient with serious cardiac insufficiency.

2. Background Art

Note that the following discussion refers to a number of publications by author(s) and year of publication, and that due to recent publication dates certain publications are not to be considered as prior art vis-à-vis the present invention. Discussion of such publications herein is given for more complete background and is not to be construed as an admission that such publications are prior art for patentability determination purposes.

Currently, implantable artificial ventricular assist devices utilize a displacement-type pump, generally driven either mechanically or electro-magnetically. Although those implantable artificial ventricular assist devices are excellent in producing physiological pulsation, they are too large for implantation in persons of small body size. While smaller implantable artificial ventricular assist devices using centrifugal or axial flow pumps exist, the production of a pulsating stream is difficult to accomplish with pumps of this design. Therefore, those pumps are used only to accomplish non-physiological continuous blood flow.

A variety of different pumps and systems which may be used for ventricular assist are disclosed in the patent literature, including those pumps and devices disclosed in U.S. Pat. Nos. 6,576,010, 6,638,011, 6,769,871, 6,866,625, and 6,884,210, and in U.S. Published Patent Application Nos. 2005/0025630 and 2004/0234397, among others. There are also disclosed a number of control systems and apparatuses which may be employed with a ventricular assist pump, including those disclosed in the foregoing patents and application and in U.S. Published Patent Application No. 2005/0014991. The foregoing patents and applications are incorporated herein by reference, it being particularly understood that certain of the control systems, components and methods may be employed with the invention disclosed herein.

The use of an “undulation pump” for pumping blood is known, including the extracorporeal artificial heart-lung apparatus disclosed in U.S. Published Patent Application No. 2004/0097861, on which the inventor is Dr. Yusuke Abe, and the pump described in Japanese Patent No. 3,285,386. Total replacement hearts utilizing an undulation pump mechanism are also known, as disclosed in Abe Y. et al., Basic Study to Develop the Undulation Pump for Practical Use: Antithrombogenicity, Hemolysis, and Flow Patterns Inside the Pump. Artificial Organs 19(7):691-693, 1995; Abe Y. et al., Development of the Undulation Pump Total Artificial Heart. Artificial Organs 21(7):665-669, 1997; Abe Y. et al., Present Status of the Total Artificial Heart at the University of Tokyo. Artificial Organs 23(3)221-228, 1999; Abe Y. et al., Development of a Miniature Undulation Pump for the Distributed Artificial Heart. Artificial Organs 24(8):656-658, 2000; Abe Y. et al., A Step Forward for the Undulation Pump Total Artificial Heart. J Artif Organs 3:70-74, 2000; Abe Y. et al., Progress in the Development of the Undulation Pump Total Artificial Heart. Journal of Congestive Heart Failure and Circulatory Support 1(4):167-170, 2001; Abe Y. et al., Third Model of the Undulation Pump Total Artificial Heart. ASAIO Journal 49:123-127, 2003; and, Abe Y. et al., Advance in Animal Experiments with the Undulation Pump Total Artificial Heart: 50 and 54 Day Survival Periods with 1/R Control. ASAIO Journal 49:325-332, 2003. The foregoing patent application, patent and articles are incorporated herein by reference.

However, there still remains a need for a long-term use and durable pump, preferably a pulsatile pump, which is implantable in a person of small stature, including women and children, for use as a ventricular assist device. It is against this background that the invention was made.

### BRIEF SUMMARY OF THE INVENTION

The present invention provides a ventricular assist undulation pump including a toroidal-shaped pump chamber with two angled side walls, an arc-shaped outer wall, an inlet port, an outlet port, and an inner circumferential opening; an undulation disk with a diameter that extends to about the arc-shaped outer wall of the pump chamber disposed within the pump chamber; and a circumferential, flexible inner wall membrane covering at least one surface of the undulation disk and forming liquid-tight seals to the pump chamber. The pump further includes a precession assembly with inner bearings connected in series with an anti-rotation assembly, the anti-rotation assembly disposed within the pump and connected to the undulation disk, and a motor in connected communication with the precession assembly so that the disk undulates when motive force is applied.

The undulation disk preferably includes a top surface and a bottom surface, and the circumferential, flexible inner wall membrane covers both the top surface and the bottom surface. The anti-rotation assembly preferably includes a universal joint and bushings.

The circumferential, flexible inner wall membrane preferably is made from a polymeric material, more preferably a thermoplastic elastomer, including polyurethane or polyethylene. The pump preferably further includes an anti-thrombogenic coating disposed on the interior surfaces of the toroidal-shaped pump, the undulation disk, and the interior surfaces of the membrane. The anti-thrombogenic coating can include segmented polyurethane or 2-methacryloyloxyethyl phosphorylcholine polymer.

The motor preferably includes a rotor that is integral to the precession assembly, with a rotor disposed on a flat stator. The rotor preferably includes a soft iron core and a plurality of magnets. Preferably, at least one balancing weight is disposed in the precession assembly. Preferably, at least one heat sink is disposed in the pump. The heat sink preferably is made from materials with a high rate of heat transfer, including aluminum, aluminum alloys, or duralumin.

The disk is made from a rigid material, preferably a metal selected from the group consisting of titanium, titanium alloys, stainless steel, aluminum and aluminum alloys. Alternatively, rigid polymeric materials may be employed to make the disk.

The present invention also provides a ventricular assist undulation pump that includes a toroidal-shaped pump chamber with two angled side walls, an arc-shaped outer wall, an inlet port, an outlet port, and an inner circumferential opening; an undulation disk with a diameter that extends to about the arc-shaped outer wall of the pump chamber disposed within the pump chamber; a circumferential, flexible inner wall membrane forming liquid-tight seals to the undulation disk and the pump chamber; a precession assembly with inner bearings connected in series with an anti-rotation assembly, the anti-rotation assembly disposed within the pump and connected to the undulation disk; and a motor in connected communication with the precession assembly so that the disk undulates when motive force is applied. The undulation disk includes a top surface and a bottom surface, and the circumferential, flexible inner wall membrane covers both the top surface and the bottom surface of the disk.

The invention also provides a ventricular assist device and system including a surgically implantable undulation pump with an inlet port and an outlet port and an implantable control unit which includes a motor drive circuit, and internal battery, a battery charge control system and an information transfer system. Preferably, the invention further includes an extracorporeal system, the system including a battery and a monitoring system. The undulation pump includes a toroidal-shaped pump chamber with two angled side walls, an arc-shaped outer wall, an inlet port, an outlet port, and an inner circumferential opening; an undulation disk with a diameter that extends to about the arc-shaped outer wall of the pump chamber disposed within the pump chamber; and a circumferential, flexible inner wall membrane covering at least one surface of the undulation disk and forming liquid-tight seals to the pump chamber. The undulation pump further includes a precession assembly with inner bearings connected in series with an anti-rotation assembly, the anti-rotation assembly disposed within the pump and connected to the undulation disk, and a motor in connected communication with the precession assembly so that the disk undulates when motive force is applied.

The present invention also provides method for assisting the ventricular circulation of blood in a patient, the method including providing a surgically implantable undulation pump with an inlet port and an outlet port, and implanting the undulation pump in the patient, with a ventricle in fluidic contact with the inlet port of the undulation pump and an artery in fluidic contact with the outlet port.

The method preferably includes providing a source of electrical power to the undulation pump, whereby the undulation pump causes blood to flow. The method preferably further includes providing an implantable control unit with includes a motor drive circuit, and internal battery, a battery charge control system and an information transfer system. The method preferably further includes providing an extracorporeal system including a battery and a monitoring system.

The undulation pump employed in the method preferably includes a toroidal-shaped pump chamber with two angled side walls, an arc-shaped outer wall, an inlet port, an outlet port, and an inner circumferential opening; an undulation disk with a diameter that extends to about the arc-shaped outer wall of the pump chamber disposed within the pump chamber; and a circumferential, flexible inner wall membrane covering at least one surface of the undulation disk and forming liquid-tight seals to the pump chamber. The pump further preferably includes a precession assembly with inner bearings connected in series with an anti-rotation assembly, the anti-rotation assembly disposed within the pump and connected to the undulation disk, and a motor in connected communication with the precession assembly so that the disk undulates when motive force is applied.

It is an object of the invention to provide an implantable ventricular assist pump, power source and control and information systems, which pump, source and system are of a sufficiently small scale to be implantable in a person of small stature.

It is another object of the invention to provide a small-sized implantable ventricular assist pump which provides pulsatile flow.

It is yet another object of the present invention to provide a small-sized implantable ventricular assist pump which both by its design and by means of appropriate coatings is anti-thrombogenic.

It is yet another object of the present invention to provide a small-sized implantable ventricular assist pump which utilizes an undulation pump wherein the flexible inner membrane is integrated with and forms a part of the blood-contacting surfaces of the undulation disk, thereby increasing safety and durability and decreasing thrombogenicity.

Other objects, advantages and novel features, and further scope of applicability of the present invention are set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated into, and form a part of, the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 shows an embodiment of the pump and system of the present invention in a patient;

FIG. 2 is an exploded, perspective view of a preferred embodiment of a pump of the present invention;

FIG. 3 is a cross-section side view of a preferred embodiment of a pump of the present invention;

FIG. 4 is a top view of a universal joint of a preferred embodiment of a pump of the present invention;

FIG. 5 is cross-section side view of a universal joint and precession assembly of a preferred embodiment of a pump of the present invention; and

FIG. 6 is a graph showing flow rates accomplished with a pump of the present invention.

### DETAILED DESCRIPTION OF THE INVENTION

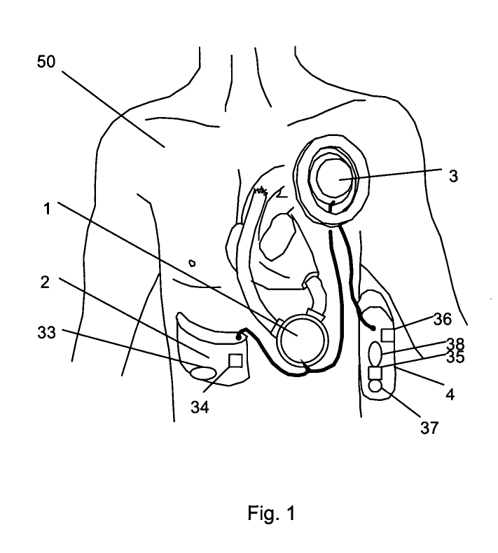

The present invention provides an implantable, artificial ventricular assist device and system. As shown in FIG. 1, the system includes implantable, artificial ventricular assist device 1; implantable controller 2 including motor drive and flow control circuit 33 and internal battery 34; transdermal energy transport system 3; and extracorporeal system 4 including battery 35, monitoring system 36, battery charge control system 37, and information transfer system 38. Ventricular assist device 1 is considerably smaller relative to similar devices in the prior art and provides a physiologically consistent, pulsation blood stream flow.

Turning to the figures, which depict a preferred embodiment of the present invention, ventricular assist device 1 includes undulation pump 5. Undulation pump 5 is characterized, in part, in that it is smaller and more efficient than the pump described in Japanese Patent No. 3,285,386, incorporated herein by reference. Ventricular assist device 1 also comprises drive assembly 6 and motor 7, hereafter described in greater detail.

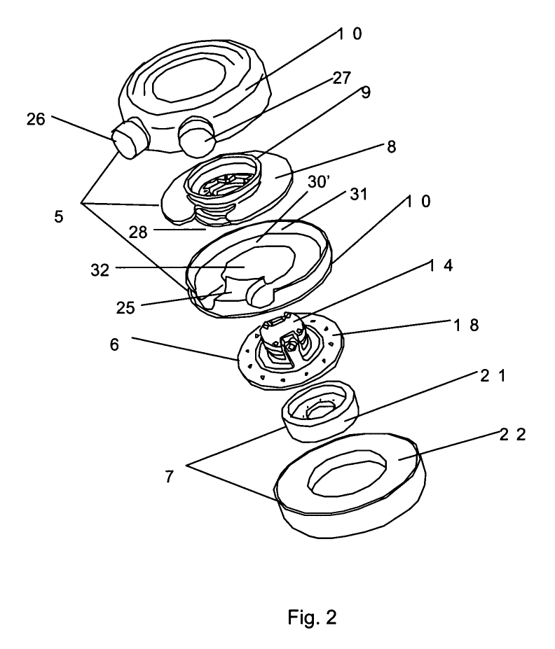

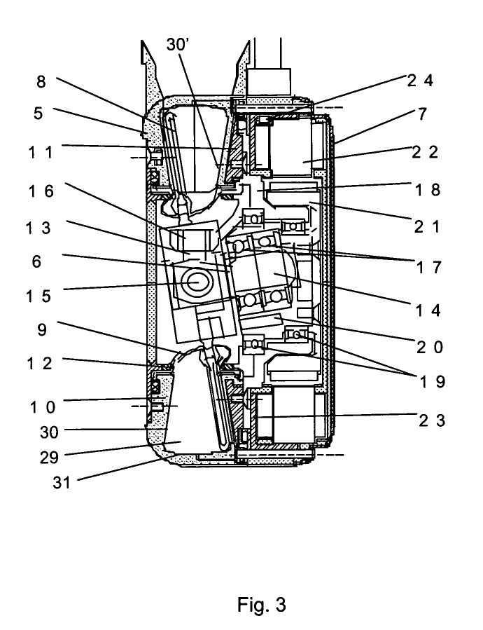

As shown in FIGS. 2 and 3, undulation pump 5 comprises two-part housing 10 including partition 25 at one end with inlet port 26 and outlet port 27 adjacent partition 25. Housing 10 forms toroidal-shaped pump chamber 29 which has two angled side walls 30, 30′, arc-shaped outer wall 31, inner, circumferential opening 32, and partition 25.

Undulation disk 8 is disposed within housing 10 and includes gap 28 providing clearance for partition 25. The diameter of undulation disk 8 extends to about arc-shaped outer wall 31 of pump chamber 29. Disk 8 is made of a rigid material, preferably a metal selected from the group consisting of titanium, titanium alloys, stainless steel, aluminum and aluminum alloys. However, alternative materials which provide sufficient rigidity and durability may be employed, including fiberglass, carbon fiber or carbon composite materials, or polymeric materials, such as, for example, polybutyrene terephthalate, polyoxymethylene, polypropylene or thermoplastic polyurethane. In general, metals are preferred due to greater manufacturing precision and stability of the shape and size over time and different ambient conditions.

Undulation disk 8 is preferably coated on all, or substantially all, blood-contacting surfaces, including the top and bottom surfaces within housing 10, with a thermoplastic elastomer membrane, which thermoplastic elastomer further forms flexible inner membrane wall 9. Most preferably, the membrane forming a coating on the blood-contacting surfaces of undulation disk 8 is continuous with inner wall membrane 9, and thus the coating on undulation disk 8 and membrane wall 9 form a single piece. The thermoplastic elastomer forming membrane wall 9 and the coating on disk 8 is of a suitable thickness to provide an acceptable life and mean time to failure for membrane 9, it being understood that in operation of pump 5 the membrane 9 is continuously flexed or moved in a wave-like motion during undulation of disk 8 during operation of pump 5. In one embodiment, the thermoplastic elastomer is a polyurethane material approximately 0.5 mm thick. However, the thickness depends on a variety of factors relating to properties of the thermoplastic elastomer, including strength, flexibility, and the like.

In previous undulation pumps, the disk transited a membrane, and thus a liquid-tight seal was required between the disk and the membrane. This seal area between the disk and membrane provided a locus for possible failure, with attendant blood leaks. By integrating the membrane with a coating on the disk, the need for a separate liquid-tight seal is eliminated, thereby providing greater reliability and patient safety.

The coating on undulation disk 8 and membrane wall 9 forming a single piece is preferably made by a process facilitating single piece construction, most preferably using injection molding techniques. Thus, in a preferred embodiment the coating on undulation disk 8 as well as the membrane wall 9 are made by injection molding. The thermoplastic elastomer preferably covers all the blood-contacting surfaces of disk 8, including the top and bottom surfaces of disk 8 as well as the exterior edge that extends proximate to about arc-shaped outer wall 31. It is to be understood that the exterior edge of disk 8, optionally and preferably with a thermoplastic elastomer coating, is sufficiently close to arc-shaped outer wall 31 to provide sufficient pumping action without unacceptable leaking or flow-back through the distance therebetween, but still without providing unacceptable friction or dragging. By utilizing a single piece coating on all blood-contacting surfaces of disk 8 which also forms membrane wall 9 it may be seen that the number of seals is half that required in the prior art. In the prior art a pair of membranes is required, one on each side of the disk, with each membrane necessarily sealed to the disk and to the housing. By providing a continuous membrane covering the disk the number of seals is half, in that seals to the disk are no longer required.

In an alternative embodiment, a portion of membrane wall 9 is continuous and of a single piece with the coating on the adjacent surface of disk 8, and the remaining portion of membrane wall 9 is continuous and of a single piece with the coating on the opposite surface of disk 8. In this embodiment, the thermoplastic elastomer employed for membrane wall 9 and the coating may similarly be made by a single-step fabrication method, such as injection molding.

The thermoplastic elastomer is, in one preferred embodiment, a polyurethane material. Alternatively, it may be a polyethylene material. In yet another embodiment, the theromoplastic elastomer is made of a material including ester elastomers, ether elastomers, polycarbonate elastomers, fluorine-containing elastomers, vinyl chloride elastomers, or styrene elastomers.

The top and bottom ends of membrane 9 are continuously and circumferentially fixed to each part of two-part housing 10, thereby forming a seal to inner, circumferential opening 32. Thus, a liquid-tight seal to pump chamber 29 is formed. In one preferred embodiment, the top and bottom ends of membrane 9 are fixed to the interior of two-part housing 10 using ring 12, and bonded using an appropriate solvent, which solvent depends, in part, upon the specific thermoplastic elastomer employed for membrane 9 as well as the material from which housing 10 is made.

After injection molding, and either prior to or after fixing membrane 9 to housing 10, the blood contacting surface of disk 8 and membrane 9 (i.e., facing chamber 29) are preferably coated with an anti-thrombogenic material, as hereafter described.

Housing 10 may be made from a medical grade polymeric material, such as polyurethane, and is preferably constructed via evacuation molding. An anti-thrombogenic material is preferably disposed on the blood-contacting interior surfaces of pump 5 (i.e., side walls 30, 30′, arc-shaped outer wall 31, and partition 25). Thus the entire interior blood-contacting surfaces of pump 5, including the thermoplastic elastomer forming a coating on disk 8 and further forming membrane 9, are coated with an anti-thrombogenic material. In one preferred embodiment, the anti-thrombogenic coating is an approximately 20 micron thick layer of an anti-thrombogenic material, such as segmented polyurethane or 2-methacryloyloxyethyl phosphorylcholine (MPC) polymer.

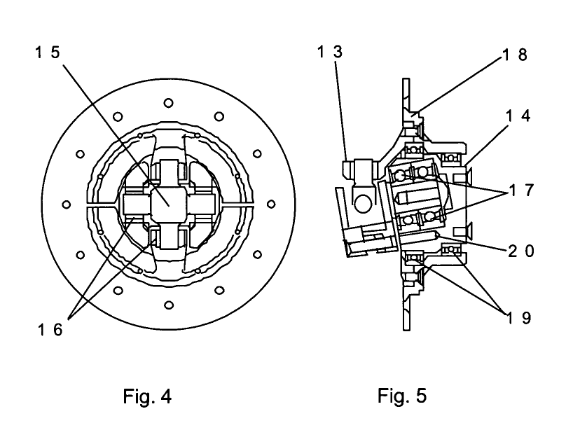

As shown in FIG. 3, drive assembly 6 includes precession assembly 14, includes inner bearings 17, and anti-rotation assembly 13 to which precession assembly 14 is connected in series. Anti-rotation assembly 13 is disposed within pump 5 and is connected to undulation disk 8. Anti-rotation assembly 13 preferably includes universal joint 15 and bushings 16. As detailed in FIGS. 4 and 5, bushings 16 are arranged along two axis oriented perpendicular to each other at the center of universal joint 13, bushings 16 of one axis fixed to disk 8, and those bushings 16 of the other axis fixed to housing 10. At least one balancing weight 20, preferably comprising tungsten, is disposed in precession assembly 14 to obtain dynamic balance.

In previous undulation pumps, the mechanisms for nutation or undulation and for prevention of rotation of the disk were concentrically disposed at the center of the undulation pump. This design required a larger space at the center of the pump. In previous undulation pumps, a ring was employed to eliminate rotational movement, but it was determined that this mechanism was not sufficiently durable and resulted in an unacceptable mean time to failure. With the design provided herein, the shaft is of smaller diameter and the precession and rotation prevention mechanisms are separate. In may thus be seen that in this design only the mechanism for prevention of rotation remains at the center of the undulation pump.

As shown in FIGS. 3 and 4, motor 7 is in connected communication with precession assembly 14 and comprises flat stator 22, which is connected to undulation pump 5, and rotor 21, which is disposed on stator 22 and is partially covered with pedestal 18, forming one body with precession assembly 14 so that rotor 21 is integral to precession assembly 14. Rotor 21 preferably comprises a core of soft iron and bonded Nd—Fe—B (neodymium-iron-boron) magnets, and is preferably coated with nitrogen titanium for rust prevention. At least one heat sink 11 is disposed adjacent stator 22. Temperature sensor 24 also is disposed adjacent stator 22. Preferably, heat sink 23 contacts with pedestal 18 which contacts with heat sink 11 of housing 10 through which blood flows. Thus, the cooling of motor 7 is provided by heat transmission from stator 22 to the blood stream. Heat sinks 11 and 23 may be made of aluminum, aluminum alloys, or duralumin. Heat sinks 11 and 23 may further include materials to assist in heat transfer, such as silicon oil.

Therefore, motor 7 is in connected communication with precession assembly 14 so that disk 8 undulates via a swinging or waving motion when motive force is applied, thus moving fluid as disk 8 undulates. By controlling the rotation of motor 7, undulation pump 5 can generate a physiologically consistent pulsating flow.

Specifically, the rotation of motor 7 is converted to motion of precession assembly 14 via bearing 17. The motion of precession assembly 14 is converted to motion of disk 8 via universal joint 13. Precession assembly 14 is held in free rotation via bearing 19 disposed in pedestal 18, which is fixed to housing 10.

Ventricular blood flow in patient 50 (shown in FIG. 1) is assisted by implanting undulation pump 5 in patient 50, with a ventricle of the heart in fluidic contact with inlet port 26 of undulation pump 5 and an artery in fluidic contact with outlet port 27 of undulation pump 5. Providing a source of electrical power, such as, for example, internal battery 34 of implantable controller 2, to undulation pump 5 causes blood to flow.

In accordance with the present invention, ventricular assist device 1 is made smaller by making the drive assembly smaller. This is accomplished in the present invention by separating the anti-rotation mechanism from the precession assembly and disposing them such that only the anti-rotation is disposed at the center of the undulation pump. Size reduction is also accomplished by making the motor flatter and smaller. This is accomplished by constructing the precession mechanism and motor rotor as one integral body. Also, the stator is made flat. A considerable reduction in noise is achieved by placing all bearings and precession mechanisms inside the drive assembly. For any number of reasons, including the psychological comfort of the patient, it is necessary to reduce the noise of any ventricular assist device as much as possible. The shortcomings of poor durability and noise that have been experienced with the use of multiple gears or with the use of a universal joint using a ring and bearings are overcome by using a universal joint comprising a cross unit and bushings.

Internal battery 34 may be any battery employed for implantable medical devices, such as a rechargeable lithium ion battery. Recharging may be effected employing extracorporeal system 4 with battery 35 and battery charge control system 37, such as by means of transdermal energy transport system 3. Transdermal energy transport system 3 may employ a high frequency, such as about 100 kHz, trans-skin transformer with two coils. Monitoring system 36 may communicate with implantable controller 2 by means such as a wireless information transfer system employing radio wave transmission. It may readily be seen that by means of the motor drive and flow control circuit 33 the rate of pumping may be varied as required for optimal patient care, and may be adjusted such that the pulse rate from the pump approximates the pulse rate of the patient's heart.

Thus, the present invention accomplishes and provides for miniaturization of an implantable artificial ventricular assist device that is suitable for use in a patient of small build, while providing a simple operating mechanism with a long life, and further providing a pulsed flow of blood.

### EXAMPLE

An implantable ventricular assist device was constructed in accordance with an embodiment of the present invention. The ventricular assist device had dimensions of approximately 69 mm in diameter and approximately 35 mm in height.

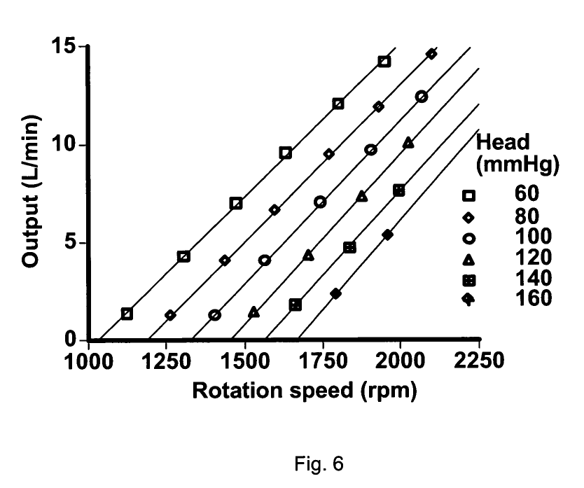

As shown in FIG. 6, a maximum blood flow rate of 13 liter per minute in continuous flow mode was achieved under a normal blood pressure. The implantable artificial ventricular assist device possessed sufficient capacity under a pulsating mode operation. Without having to administer anti-thrombosis therapy, no blood clot formation was observed in animal tests during a period of 63 days of continuous operation of the device which employed a segmented polyurethane coating on all the blood contacting surfaces.

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above, and of the corresponding application(s), are hereby incorporated by reference.

1. A ventricular assist undulation pump comprising:
a toroidal-shaped pump chamber with two angled side walls, an arc-shaped outer wall, an inlet port, an outlet port, and an inner circumferential opening;
an undulation disk with a diameter that extends to about the arc-shaped outer wall of the pump chamber disposed within the pump chamber; and
a circumferential, flexible inner wall membrane covering the inner circumferential opening and at least one surface of the undulation disk and forming liquid-tight seals to the pump chamber.

2. The pump of claim 1, further comprising:
a precession assembly with inner bearings connected in series with an anti-rotation assembly, the anti-rotation assembly disposed within the pump and connected to the undulation disk; and
a motor in connected communication with the precession assembly so that the disk undulates when motive force is applied.

3. The pump of claim 1 wherein the undulation disk comprises a top surface and a bottom surface, and wherein the membrane covers both the top surface and the bottom surface.

4. The pump of claim 2 wherein the anti-rotation assembly comprises a universal joint and bushings.

5. The pump of claim 1 wherein the membrane comprises a material selected from the group consisting of polyurethane and polyethylene.

6. The pump of claim 1 wherein the membrane comprises a thermoplastic elastomer.

7. The pump of claim 1, further comprising an anti-thrombogenic coating disposed on the blood-contacting surfaces of the toroidal-shaped pump chamber, the undulation disk, and the interior surfaces of the membrane.

8. The pump of claim 7 wherein the anti-thrombogenic coating comprises segmented polyurethane.

9. The pump of claim 7 wherein the anti-thrombogenic coating comprises 2-methacryloyloxyethyl phosphorylcholine polymer.

10. The pump of claim 2 wherein the motor comprises a rotor that is integral to the precession assembly, the rotor disposed on a flat stator.

11. The pump of claim 10 wherein the rotor comprises a soft iron core and a plurality of magnets.

12. The pump of claim 2, further comprising at least one balancing weight disposed in the precession assembly.

13. The pump of claim 8, further comprising at least one heat sink disposed in the pump.

14. The pump of claim 13 wherein the at least one heat sink comprises a metal selected from the group consisting of aluminum, aluminum alloys, and duralumin.

15. The pump of claim 1 wherein the disk comprises a rigid material.

16. The pump of claim 15 wherein the disk comprises a metal selected from the group consisting of titanium, titanium alloys, stainless steel, aluminum and aluminum alloys.

17. A ventricular assist undulation pump comprising:
a toroidal-shaped pump chamber with two angled side walls, an arc-shaped outer wall, an inlet port, an outlet port, and an open circumferential inner wall;
an undulation disk with a diameter that extends to about the arc-shaped outer wall of the pump chamber disposed within the pump chamber;
a circumferential, flexible inner wall membrane covering the inner circumferential opening and forming liquid-tight seals to the undulation disk and the pump chamber;
a precession assembly with inner bearings connected in series with an anti-rotation assembly, the anti-rotation assembly disposed within the pump and connected to the undulation disk; and
a motor in connected communication with the precession assembly so that the disk undulates when motive force is applied.

18. The pump of claim 17 wherein the undulation disk comprises a top surface and a bottom surface, and wherein the membrane covers both the top surface and the bottom surface.

19. The pump of claim 17 wherein the anti-rotation assembly comprises a universal joint and bushings.

20. The pump of claim 17 wherein the membrane comprises a material selected from the group consisting of polyurethane and polyethylene.

21. The pump of claim 17 wherein the membrane comprises a thermoplastic elastomer.

22. The pump of claim 17, further comprising an anti-thrombogenic coating disposed on the interior surfaces of the toroidal-shaped pump chamber, the undulation disk, and the interior surfaces of the membrane.

23. The pump of claim 22 wherein the anti-thrombogenic coating comprises segmented polyurethane.

24. The pump of claim 22 wherein the anti-thrombogenic coating comprises 2-methacryloyloxyethyl phosphorylcholine polymer.

25. The pump of claim 17 wherein the motor comprises a rotor that is integral to the precession assembly, the rotor disposed on a flat stator.

26. The pump of claim 25 wherein the rotor comprises a soft iron core and a plurality of magnets.

27. The pump of claim 17, further comprising at least one balancing weight disposed in the precession assembly.

28. The pump of claim 25, further comprising at least one heat sink disposed in the pump.

29. The pump of claim 28 wherein the at least one heat sink comprises a metal selected from the group consisting of aluminum, aluminum alloys, and duralumin.

30. The pump of claim 17 wherein the disk comprises a rigid material.

31. The pump of claim 30 wherein the disk comprises a metal selected from the group consisting of titanium, titanium alloys, stainless steel, aluminum and aluminum alloys.

32. A ventricular assist device comprising:
a surgically implantable undulation pump with an inlet port and an outlet port; and
an implantable control unit comprising a motor drive circuit, and internal battery, a battery charge control system and an information transfer system.

33. The device of claim 32, further comprising an extracorporeal system comprising a battery and a monitoring system.

34. The device of claim 32, wherein the undulation pump comprises:
a toroidal-shaped pump chamber with two angled side walls, an arc-shaped outer wall, an inlet port, an outlet port, and an inner circumferential opening;
an undulation disk with a diameter that extends to about the arc-shaped outer wall of the pump chamber disposed within the pump chamber; and
a circumferential, flexible inner wall membrane covering the inner circumferential opening and at least one surface of the undulation disk and forming liquid-tight seals to the pump chamber.

35. The device of claim 34, wherein the undulation pump further comprises:
a precession assembly with inner bearings connected in series with an anti-rotation assembly, the anti-rotation assembly disposed within the pump and connected to the undulation disk; and
a motor in connected communication with the precession assembly so that the disk undulates when motive force is applied.

36. A method for assisting the ventricular circulation of blood in a patient, comprising:
providing a surgically implantable undulation pump with an inlet port and an outlet port; and
implanting the undulation pump in the patient, with a ventricle of the patient's heart in fluidic contact with the inlet port of the undulation pump and an artery of the patient in fluidic contact with the outlet port.

37. The method of claim 36, further comprising providing a source of electrical power to the undulation pump, whereby the undulation pump causes blood to flow.

38. The method of claim 36, further comprising providing an implantable control unit comprising a motor drive circuit, and internal battery, a battery charge control system and an information transfer system.

39. The method of claim 38, further comprising providing an extracorporeal system comprising a battery and a monitoring system.

40. The method of claim 36, wherein the undulation pump comprises:
a toroidal-shaped pump chamber with two angled side walls, an arc-shaped outer wall, an inlet port, an outlet port, and an inner circumferential opening;
an undulation disk with a diameter that extends to about the arc-shaped outer wall of the pump chamber disposed within the pump chamber; and
a circumferential, flexible inner wall membrane covering the inner circumferential opening and at least one surface of the undulation disk and forming liquid-tight seals to the pump chamber.

41. The method of claim 40, wherein the undulation pump further comprises:
a precession assembly with inner bearings connected in series with an anti-rotation assembly, the anti-rotation assembly disposed within the pump and connected to the undulation disk; and
a motor in connected communication with the precession assembly so that the disk undulates when motive force is applied.
